(19) United States
(12) Patent Application Publication
(10) Pub. No.: US 2024/0230617 A9
(43) Pub. Date: Jul. 11, 2024

(54) PROCESS TO EXTRACT AND DETERMINE NITROGEN COMPOUNDS AND ACIDS PRESENT IN PETROLEUM, DERIVATIVES AND PROCESSING WATERS BEFORE THE REFINING STEP

(71) Applicants: PETRÓLEO BRASILEIRO S.A. - PETROBRAS, Rio de Janeiro (BR); SERVIÇO NACIONAL DE APRENDIZAGEM INDUSTRIAL SENAI, Rio de Janeiro (BR)

(72) Inventors: ROGERIO MESQUITA DE CARVALHO, Rio de Janeiro (BR); SERGIO NOBORU KURIYAMA, Rio de Janeiro (BR); ANDRE LUIZ CASTRO BONFIM, Rio de Janeiro (BR); ANTÔNIO AUGUSTO FIDALGO NETO, Rio de Janeiro (BR); SAMUEL DE OLIVEIRA SILVA, Rio de Janeiro (BR); GIOVANNI BRESSIANI PEDROSO, Rio de Janeiro (BR)

(73) Assignees: PETRÓLEO BRASILEIRO S.A. - PETROBRAS, Rio de Janeiro (BR); SERVIÇO NACIONAL DE APRENDIZAGEM INDUSTRIAL SENAI, Rio de Janeiro (BR)

(21) Appl. No.: 18/489,132

(22) Filed: Oct. 18, 2023

(30) Foreign Application Priority Data
Oct. 19, 2022 (BR) BR1020220212350

(51) Int. Cl. G01N 33/28

(52) U.S. Cl. CPC G01N 33/2835

(57) ABSTRACT

The present invention proposes a process for extracting and determining nitrogenous and acidic compounds in various types of petroleums, derivatives or thermal degradation products, and in process waters before the production/refining phase.

When the methodology is applied to petroleum and byproducts, the process comprises the extraction steps (1); separation (2); pre-concentration (3); and analysis (4).

When the methodology is applied to thermal degradation products, there is a need for a previous step to simulate the heating of petroleum containing different chemicals (if necessary) with the purpose of promoting thermal degradation of the species present and of the petroleum, and generating nitrogen compounds with simpler composition.

### FIELD OF THE INVENTION

The present invention belongs to the technical field of supply and biofuels, and more specifically, to the field of coke and separation, distribution, logistics and transport processes, hydrorefining and treatments. It can also be used in the field of sustainable development, and more specifically, in water treatment and reuse, as well as in petroleum production processes, lifting and flow technologies and processing technologies primary.

### BACKGROUND OF THE INVENTION

The management of oilfield produced water constitutes an enormous challenge for petroleum companies as it is a very relevant by-product due to its high volume and inevitable role in the petroleum extraction, production and refining chain. The alternatives usually adopted for its destination are disposal, injection and reuse in the process.

In all cases, it is necessary to treat oilfield produced water in order to avoid damage to the environment and production facilities, or to allow its reuse without causing harm to the processes in which the oilfield produced water will be used.

The study of its composition and the understanding that certain operations add compounds to this system are extremely relevant. However, to this day, there remains a demand for the development of robust methodologies in the oil and gas field that allow the qualification and quantification of compounds in the oily matrix, which exhibit affinity for and partition into the aqueous phase.

The few studies that, nowadays, report analytical initiatives are focused on chemical classes that are applied in the North Sea and, therefore, have relevant chemical differences that prevent the simple reproduction of this procedure within the reality of Brazilian production.

Analysis metrics such as titratable acidity (TAN) do not provide an answer to the extraction challenge, and instrumental techniques, such as chromatography and spectroscopy, can perform speciation, but still require intensive sample preparation.

Therefore, there are not integrated processes intended for handling the oily matrix and subsequent quantification with regard to carboxylic acids.

In particular, for the classes of nitrogenous compounds present in fuel residues, their poisoning nature in the cracking and refining process is recognized (for example, solid deposits in fractional distillation towers used during the refining process), in addition to several environmental problems. Due to their high complexity, the characterization of alkaline nitrogen compounds (such as pyridine structures) and neutral nitrogen compounds (in pyrrole structures) is not feasible using a simple protocol. Furthermore, nitrogenous species of simpler composition can be formed from thermal degradation during the distillation procedure and impact the formation of salts in the top region of the distillation tower. This fact becomes even more complex in the presence of chemical inputs that scavenge nitrogenous H₂S. Additionally, carboxylic acids are also present in the oil (mainly in crude, immature and biodegraded oils, fractions and heavy oils, as well as in process waters), being composed of naphthenic structures between 8 and 20 carbons, containing saturated rings and variable side alkyl chain. Occasionally, acids with a linear or aromatic chain structure may also be present.

This, in short, this fraction includes a varied range of compounds that also incur operational problems and challenges, and mainly the formation of stable emulsions of the oil-in-water (and also water-in-oil) type, the increase in the oil and grease content in disposal, corrosion in refining units, increased operating costs for effluent treatment, in addition to buffering effects and the release of oxidative species into the environment.

To avoid these problems, some processes for extracting organic and naphthenic acids have already been proposed using previous techniques, but none involve the methodology developed in the present invention, both by the logic of extraction from the oil and by the sequence of process and preparation steps, and analysis of the extracted aqueous fraction.

Document PI 9909116-0, from Exxon Research and Enginnering, 1998, teaches a process for extracting organic acids, including naphthenic acids, heavy metals and sulfur from a starting crude oil comprising the steps of (a) treating the starting crude oil containing organic acids, heavy metals and sulfur with an amount of ethoxylated amine and water for a period of time and temperature sufficient to form a water-in-oil emulsion of an amine salt; (b) separating the emulsion from step (a) into several layers, where one of these layers contains a treated crude oil that has decreasing amounts of organic acids, heavy metals and sulfur; (c) recovering the layer from step (b) containing the treated crude oil and with decreasing amounts of organic acids, heavy metals and sulfur and the layers containing water and the ethoxylated amine salt.

Document PI 9909182-0, from Vetco Gray Scandinavia AS, from 1998, teaches a process very similar to that of PI9909116-0; however it uses alkoxylated amine to form a water-in-oil emulsion of an amine salt.

U.S. Pat. No. 4,752,381, from Nalco Chemical Company (already in the public domain), teaches a process for neutralizing organic acidity in petroleum and petroleum fractions to provide a neutralization number less than 1.0. The process treats the petroleum fraction with a monoethanolamine to form an amine salt followed by heating, for a sufficient period of time and temperature to form an amide. These amines do not provide the results desired in the present invention, since they convert naphthenic acids into other products, while the present invention extracts naphthenic acids.

Document U.S. Pat. No. 4,477,337 (which is a continuation of the abandoned U.S. Pat. No. 397,935), from Husky Oil Operations Ltd. 1983, teaches a method for removing solids and water from crude petroleum. The document addresses a method capable of reducing the water and sediment content (BSW) of a wide range of heavy oil flood and steam flood crudes to an acceptable level. The document is concerned with the removal of sulfur and other solids and not with the removal of acids and nitrogenous compounds.

Document U.S. Pat. No. 5,080,779, from Betz Laboratories, 1992, teaches a method for removing iron from crude petroleum, but is not concerned with the removal of acids and nitrogenous compounds.

U.S. Pat. No. 7,750,302, owned by Schlumberger Technology, refers to methods and apparatus for detecting the presence and/or measuring the amounts of naphthenic acids in formation fluids, particularly in effluents from hydrocarbon reservoirs. According to the document, a method for determining the concentration of organic acids in formation fluids is provided using the steps of (i) pumping fluids from an underground formation into the body of a downhole tool and (ii) illuminating the flow with infrared radiation to obtain an infrared absorption or a related parameter at one or more wavelengths, and convert the measured absorption to the concentration of organic acids, using a multi-valued calibration matrix that links IR absorption spectral values to concentration measurements under downhole conditions. These downhole naphthenic acid measurements are then correlated to estimate the total acid number (TAN) of the hydrocarbon oil produced from the formation. TAN can be used in the refining or downstream industry as a parameter to determine the commercial value of the petroleum produced or as a parameter to determine the further processing of crude petroleum. However, the document does not address the extraction/removal of organic acids from crude petroleum.

Document WO 20A2, from ExxonMobil Research and Engineering, teaches a method for reducing the naphthenic acid content of crude petroleum and its refinery stream fractions by contacting a stream of crude petroleum or petroleum distillate in the presence of an effective amount of water, a base selected from Group IA and IIA hydroxides and ammonium, and a phase transfer agent at an effective temperature for a time sufficient to produce a treated petroleum feed with a decreased naphthenic acid content and an aqueous phase containing naphthenate salts, phase transfer agent and base.

Advantageously, this process facilitates the extraction of higher molecular weight naphthenic acids (in addition to lower molecular weight naphthenic acids), which would otherwise remain in the petroleum stream after caustic soda extraction. This results in lower TAN content and reduced corrosion of refinery equipment. Furthermore, the presence of the phase transfer agent reduces emulsion formation after caustic treatment, and this leads to better processability. Despite referring to a process for extracting naphthenic acids, the process is different from that addressed in the present invention.

Document U.S. Pat. No. 5,182,013, from Exxon Chemical, 1990, teaches a process for inhibiting corrosion caused by naphthenic acid in refining operations. The document proposes mixing an oil with a high naphthenic acid content with an oil with a low naphthenic acid content. Additionally, several attempts have been made to solve the problem by using corrosion inhibitors on metal surfaces of equipment exposed to acids, or by neutralizing and removing acids from oil. However, the document does not address the extraction/removal of organic acids from crude petroleum.

Document U.S. Pat. No. 4,199,440, from UOP Inc., 1978, describes a process for removing traces of acidic compounds (carboxylic acids, H₂S, naphthenic acids, among others) from liquid hydrocarbons. Traces of these compounds are removed by injecting a dilute aqueous alkaline solution into the hydrocarbon stream and passing this stream through a coalescing bed. The document addresses a methodology different from the present invention.

Document U.S. Pat. No. 4,634,519, from Chevron Research Company, 1985, teaches a process for extracting naphthenic acids from petroleum distillates using a solvent system comprising liquid alkanols, water and ammonia in certain critical ratios to facilitate selective extraction and easy separation. Although it also presents an extraction step, the extraction is carried out in the refining step, while in the present invention, the extraction is carried out before the refining step. Therefore, the document addresses a methodology different from the present invention.

The Chinese document CN 1418934, from the Equipment Inst Luoyang Petroch, 2001, lists steps such as neutralization, addition of demulsifier and use of an electrostatic separator. Therefore, it presents a solution far from the process logic presented by the present invention.

Chinese Patent CN 111560252, from Univ China Petroleum East China, 2020, focuses on the removal of naphthenic species from soils contaminated with crude oil and addresses initiatives involving microemulsions. Although there is analysis of the target compounds, there is no analytical similarity with the technique used in the present invention.

Document US 2019/0048268, from Amperage Energy Inc., 2018, teaches the use of a caustic solution followed by atomization to remove TAN in the water/oil system. Although efficient, the objective of the work is not comparable with the present invention.

Document WO 2019/099231, from BL Technologies, Inc., 2017, describes the use of SPE for selective extraction of amines (aliphatic and short chain) from crude oil. The document does not mention subsequent analysis steps or even the use of pre-extracted samples.

Furthermore, the dissemination of scientific articles published in journals under the responsibility of the main publishers (Elsevier and Wiley) did not return works that reproduce the knowledge developed here in its entirety, but only partially.

Currently, if it is necessary to know some type of chemical species, be it an acid or a nitrogenous compound, it is mandatory to collect water from the process, which is often not possible, and depend on its availability. Furthermore, there is also no approach for evaluating species arising from thermal degradation in petroleum (with or without chemicals present).

The method developed by the present invention by previously evaluating oil samples and being able to determine nitrogenous and acidic compounds in oils allows actions to be taken in advance, considering oils that have not yet entered the production/refining phase. In other words, based on knowledge of the presence of such species, actions can be adopted in the design condition, whether in new units or in additional units that are built.

When the assessment is carried out on process waters, the methodology presents the advantages of reducing the use of acids in the chemical treatment route of species dissolved in produced water for disposal, which increases the health and safety of the process used. Furthermore, it allows the adoption of strategies to reduce the content of oils and greases (TOG) in produced water; enabling the production/disposal of produced water to the detriment of reinjection of produced water, selection of chemical inputs that result in the most appropriate breakdown of the emulsion, favoring production; evaluation of the impact of chemical inputs on petroleum refining, mainly from the point of view of the top region of the distillation tower where salt deposits can be formed, leading to clogging and the prediction of the presence of polar species that could eventually impact on the disposal of produced water, considering petroleum production in an offshore environment.

Thus, it is clear that the documents cited and commented above do not present similar studies or processes, nor the possible technical advantages of the present invention, as reported above. Therefore, it is possible to note that the state of the art lacks a process for the extraction and determination of nitrogenous compounds and acids applied to Brazilian petroleum and process waters, as detailed below.

### SUMMARY OF THE INVENTION

One of the objectives of the present invention is to evaluate and estimate the presence of species that may influence the effluent disposal parameter associated with the oil and grease content, even from oils that have not yet been produced and contain a low water content. Such measures allow the adoption of actions even at the design step (both from the point of view of new treatment or reinjection technologies), significantly reducing costs in the implementation phase. Application in evaluating the stability of emulsions and selecting the best solutions from a chemical point of view (using suitable inputs to break the emulsion). Assessment of species with corrosive potential or that may influence the processing of petroleum in the refining step and the adoption of mitigating or even minimizing actions based on the information obtained. Selection of nitrogenous products that result in less impact on the formation of salts in the distillation tower and monitoring of species that have the potential or may influence the formation of such deposits.

To this end, the present invention proposes a process for extracting and determining nitrogenous compounds and acids present in petroleum, derivatives and processing waters before the refining step, which comprises the macrosteps of:
extraction (1);
separation (2);
pre-concentration (3); and
analysis (4).

The process may also include an optional step of heating the petroleum before the extraction step when the analysis is carried out on the thermal degradation products of the petroleum.

### BRIEF DESCRIPTION OF FIGURES

FIG. 1 is a flowchart with the macrosteps of the extraction and determination process of nitrogenous and acidic compounds in oils, derivatives or thermal degradation products of the present invention.

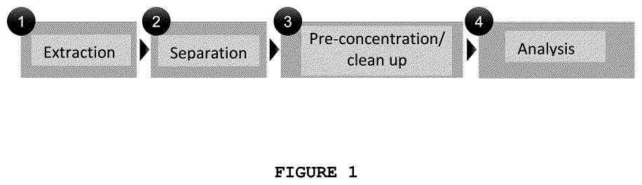

### DETAILED DESCRIPTION OF THE INVENTION

The invention proposes a process for extracting and determining nitrogenous and acidic compounds in various types of oils, derivatives or thermal degradation products, and in process waters before the production/refining phase.

When the methodology is applied to petroleum and byproducts, the process comprises the extraction steps (1); separation (2); pre-concentration (3); and analysis (4).

When the methodology is applied to thermal degradation products, there is a need for a previous step to simulate the heating of petroleum containing different chemicals (if necessary) with the purpose of promoting thermal degradation of the species present and of the petroleum, and generating nitrogen compounds with simpler composition.

Depending on the composition of the products, there may be an influence on the formation of the nitrogenous species of interest. The most typical case that results in the formation of nitrogen is the addition of H₂S scavenger.

Therefore, the methodology of the present invention is based on a sequence of steps that allows the processing of oils with different characteristics (BSW, suspended solids, acidity levels), their derivatives, and also the aqueous process fractions based on chemical analysis of the oils and/or aqueous fractions by complementary instrumental techniques.

Methodology for Extraction and Determination of Nitrogenous and Acidic Compounds in Thermal Degradation Products

The approach for generating thermal degradation products involves conducting laboratory experiments under controlled and known conditions, heating in the system to simulate the passage of petroleum containing diverse chemical compounds and their resultant reaction products in preheating batteries and atmospheric furnaces within refineries, where the oil can be heated to temperatures of up to 350° C., causing the thermal degradation of the chemical components and resulting in the generation of sulfur compounds that become concentrated in the gas phase.

a) Addition of Reaction Products of H₂S Scavengers in the Oil

Transfer 800 mL of petroleum to a metallic pressure vessel, keep under constant mechanical stirring and submit to a continuous flow of gas mixture containing H₂S at atmospheric pressure and room temperature. Continuously measure the concentration of H₂S in the effluent gas phase of the reaction system until saturation is verified, when the concentration of H₂S at the outlet becomes equal to the input. After saturation of the reaction medium with the gas mixture, add an aliquot of the H₂S scavenging chemical product to be evaluated in a pre-established dosage provided by the product manufacturer. Keep the system in this condition for 2 hours with continuous bubbling of H₂S, to promote the reaction of the scavenging chemical product with H₂S to the maximum extent possible.

b) Thermal Degradation of Oil into Chemicals

To promote thermal degradation of the samples, a 500 mL metallic pressure vessel (autoclave) made of stainless steel or nickel metal alloy, a 400 mL glass container, internal to the metallic vessel, and instrumentation for monitoring and recording the pressure and temperature, are used. Transfer 100 mL of each previously prepared oil (20% of the total volume), containing chemicals and their respective reaction products, to the glass container, place inside the pressure vessel, close the pressure vessel and submit to the pre-established temperature (350° C.). When the target temperature is reached, the system is maintained under this condition for 10 minutes.

Extraction Step

The oily samples are mechanically homogenized, usually for 10 min at a speed of ±300 rpm, and an aliquot of 100 to 250 mL is collected for extraction in a round-bottom flask.

For extraction, the system is kept under heating at 65±10° C. and constant stirring (±200 rpm) for 8 hours, with an aqueous solution in a stoichiometric ratio of 4:1 (oil/water), resulting in final volumes of 500.0 to 1,000.0 mL. The pH of the medium influences the class of compounds to be extracted. For the extraction of nitrogenous compounds, the pH conditions of the medium must be set at values below 5.0, and for the extraction of acidic compounds, above 11.0.

Separation Step

Once the extraction step is complete, the samples must be centrifuged at 9,500 rpm for 180 min, with the occasional use of demulsifier at a concentration of up to 2 mg/100 mL of sample, and collected in a separating funnel. The demulsifiers to be used are generally those based on ethylene/propylene oxide copolymers, commonly used in the petroleum industry. The oily fraction is dispensed and the aqueous fraction goes to a later step, monitoring the pH of this fraction.

If necessary, alkaline and total nitrogen can be quantified (fractions of 80.0 and 10.0 mL, respectively) in addition to the determination of acidic species by potentiometric titration (10 mL) with NaOH, in the aqueous fraction.

Pre-Concentration/Clean Up Step

The aqueous fractions proceed to percolation in a pre-conditioned solid phase extraction cartridge. Succinctly, the protocol presents three variants, which can be used individually or together, depending on the objectives:
Polymeric cation exchanger: focus on short-chain amines. It consists of conditioning with 3.0 mL of hexane; 3.0 mL of methanol; 3.0 of water (pH 5.0), sample percolation (pH 5.0), drying for 30 minutes, constant flow (1 mL/min) and elution with 2×1.5 mL of aqueous phase, pH 10.0±0.2 (with Ca(OH)₂) and 2×1.5 mL aqueous phase, pH 10.0±0.2 and acetonitrile (ratio 1:1);
Weak anion exchanger: focus on short-chain acids. It consists of conditioning with 6.0 mL of Methanol+6.0 of water (pH 2.5), sample percolation (pH 2.0), drying for 30 minutes, constant flow (1 mL/min) and elution with 2×1.5 mL of aqueous phase (pH<1.0);
C18 resin: focus on long-chain acidic or nitrogenous compounds. It consists of conditioning with 12.0 mL of methanol+12.0 mL of water (pH 2.5), sample percolation (pH 2.5), drying for 30 minutes, constant flow (2 mL/min) and elution with 2×2.5 mL of methanol or 2×2.5 mL of dicholomethane.

Analysis Step

The pre-concentrated fraction can be used for quantification by gas chromatography couplet to mass spectrometry detection (GC-MS), ion exchange chromatography with conductivity detection (IC) and spectroscopy in the infrared region (FTIR) by attenuated reflectance. The application of each analytical technique depends on the type of analyte to be analyzed.

The methodology considers the use of:
IC for analysis of short-chain amines: 4×250 mm (4 um) column, flow rate of 0.9 mL/min, 40° C., with mobile phase of 0.7 mmol/L of dipicolinic acid (1.7 mmol/L) in HNO₃; 20.0 μL injection;
IC for analysis of short-chain acids: 250×7.8 mm column, flow rate of 0.5 mL/min, 30° C., with mobile phase of 0.5 mmol/L; 10.0 μL injection;
GC-MS for analysis of long-chain compounds: RTx-5 ms column (Restek), FE 5% phenylmethyl; 95% polydimethylsiloxane (30 m×0.25 mm×0.25 μm film), 330° C. (injection) in split mode (50×), with electron impact ionization in a quadrupole detector (scan mode, 45-500 m/z);
FTIR for analysis of acidic compounds: evaporation and resuspension in heptane, reading in reflectance mode, (1.0 mL sample, reading with 45 scans, resolution of 4 cm⁻¹, ZnSe crystal), with monitoring between 1690 and 1730 cm⁻¹.

Methodology for Extraction and Determination of Nitrogenous and Acidic Compounds in Petroleum Products and Derivatives

When it is intended to obtain information regarding nitrogen compounds or acidic species that can migrate to the aqueous phase, the methodology is applied directly to petroleum and derivatives. In this specific case, the process only comprises the steps of: extraction (1); separation (2); pre-concentration (3); and analysis (4), basically the same steps already described above. For the specific case of obtaining data associated with process waters, the steps to be applied to obtain information regarding nitrogenous and acidic species are: pre-concentration (3); and analysis (4).

Example of Embodiments/Tests/Results Obtained

The extraction of nitrogenous and acidic compounds from oils, derivatives and thermal degradation products to estimate the presence of these compounds and predict possible migrations to the aqueous fraction (i.e. process water) involves an intensive process of sample handling and understanding the factors relevant to extraction, such as pH, time and concentrations involved. In addition to a better understanding of the process and taking mitigating actions, minimization actions can also be carried out, through interventions in the process or even in the design of new units. This is only possible through oil analysis, as produced water is not yet available. Thus, and in an unprecedented way, a reproducible protocol was developed and validated against systems doped with analytical standards to estimate the recovery efficiency and process reproducibility.

Briefly, the recoveries obtained are close to 100% (w/w) when the system is maintained for 8 hours, under ideal temperature conditions, and with pH above 11.0 and below 5.0, for acidic and nitrogenated, respectively.

This result is even more relevant considering that it is maintained even with pre-concentration factors of up to 50×, increasing the sensitivity of the methodology simultaneously with the elimination of interferents, through the use of the solid phase extraction (SPE) step.

Additionally, the process developed here proved to be applicable for samples with BSW of up to 10% (w/v), and even post-thermal stress protocols (>300° C., in the presence of triazine and ethoxylate), especially considering that each sample can be analyzed under different pH conditions and, therefore, have evaluated both the maximum extraction (i.e., more extreme values) and usual conditions in the field (pH between 7.0 and 8.0).

Regarding the identification and quantification of the compounds of interest in the extracted and pre-concentrated aqueous fractions, the sensitivity was around 100 mg/L. These values are consistent with quantification limits of 2 mg/L of oil, values referring to a concentration of 0.2% w/w, that is, far below the values reported in the literature (>1.0% w/ml) as generators of incidents in refining plants.

As an example, in table 1 below, there is a comparison of the analysis of total nitrogen and alkaline nitrogen obtained after all the steps described. Based on these results, it is concluded that the most suitable pH for extracting nitrogenous species is 5 or lower. Furthermore, it becomes possible to obtain data that allows the comparison and evaluation of the removal of nitrogenous species considering the aforementioned process.

Table 1: Results obtained after applying the steps of extraction, separation and pre-concentration in petroleum, considering the focus on the parameter: nitrogenous species—total and alkaline nitrogen in petroleum before the desalting process (salted) and after the desalting process (desalted).

| | salty petroleum | | | desalted petroleum | | |
|---|---|---|---|---|---|---|
| | pH < | pH = | pH = | pH < | pH = | pH = |
| Parameter | 5 | 7 | 10 | 5 | 7 | 10 |
| N ₜₒₜₐₗ (mg/kg) | 169 | 46 | 27 | 167 | 42 | 32 |
| N ₐₗₖₐₗᵢₙₑ (mg/kg) | 253 | 45 | 10 | 190 | 21 | 14 |

Another example demonstrates the application of the approach after thermal degradation in the absence and presence of different H₂S scavengers to a specific petroleum, after all the steps mentioned (1 to 4), with the analysis carried out via FTIR for acid quantification. The impact of the use of scavengers on the formation of acids is observed.

Table 2: Results obtained after applying the extraction, separation and pre-concentration steps in petroleum after thermal degradation in the absence and presence of H₂S scavenger, considering focus on the parameter: acid concentration.

| Sample | Acid concentration (mg/L) |
|---|---|
| Petroleum without scavenger | <100 |
| Petroleum with scavenger A | 336 |
| Petroleum with scavenger B | 458 |

This result, if considered as results of complementary analytical methodologies involving spectroscopy and chromatography, makes this process robust and capable of characterizing oily samples against these compounds.

1. A process to extract and determine nitrogen compounds and acids present in petroleum, derivatives and processing waters before a refining step, comprising the steps:
(a) extraction;
(b) separation;
(c) pre-concentration; and
(d) analysis.

2. The process according to claim 1, further comprising a step of heating the petroleum before the extraction step, and wherein the analysis is carried out on the thermal degradation products of the petroleum.

3. The process according to claim 1, wherein the extraction step comprises keeping a sample under heating at 65±10° C. and constant stirring at 200 rpm for 8 hours with an aqueous solution in a stoichiometric ratio of 4:1 (oil/water).

4. The process according to claim 3, wherein in the extraction step, the pH of the medium is below 5.0 for extraction of nitrogenous compounds or above 11.0 for extraction of acidic compounds.

5. The process according to claim 1, wherein after the extraction step, samples are centrifuged at 9,500 rpm for 180 min with addition of 2 mg demulsifier per 100 mL sample.

6. The process according to claim 5, wherein the demulsifiers used are based on ethylene/propylene oxide copolymers.

7. The process according to claim 5, wherein the samples are separated and collected in a separating funnel wherein the oily fraction is dispensed and the aqueous fraction goes to a subsequent step, while monitoring a pH of the aqueous fraction.

8. The process according to claim 1, wherein the aqueous fraction after the separation step is pre-concentrated by passing through three pre-conditioned solid phase extraction cartridges.

9. The process according to claim 8, wherein the 3 pre-conditioned solid phase extraction cartridges are a polymeric cation exchanger for separating short chain amines, a weak anion exchanger for separating short-chain acid compounds, and a C18 resin to separate long-chain compounds.

10. The process according to claim 9, wherein the aqueous fraction can pass through the 3 pre-conditioned solid phase extraction cartridges together or individually, depending on the intended purposes.

11. The process according to claim 10, wherein the aqueous fraction, after pre-concentration, is used for quantification by a method comprising one or more of gas chromatography coupled to mass spectrometry detection (GC-MS), ion exchange chromatography with conductivity detection (IC) and spectroscopy in the infrared region (FTIR) by attenuated reflectance, wherein the method depends on the analyte to be analyzed.

12. The process according to claim 11, wherein quantification by gas chromatography coupled to mass spectrometry detection (GC-MS) identifies long-chain compounds, ion exchange chromatography with conductivity detection analysis (IC) identifies short-chain amines and acids, and quantification by spectroscopy in the infrared region (FTIR) by attenuated reflectance identifies acidic compounds.
